(19) United States
(12) Patent Application Publication
(10) Pub. No.: US 2017/0130029 A1
(43) Pub. Date: May 11, 2017

(54) POLYSULFIDE ADDITIVE, METHOD FOR PRODUCTION THEREOF AND USE THEREOF IN RUBBER MIXTURES

(71) Applicant: LANXESS Deutschland GmbH, Cologne (DE)

(72) Inventors: Ulrich FELDHUES, Bergisch Gladbach (DE); Heinz UNTERBERG, Dormagen (DE); Hermann-Josef WEIDENHAUPT, Pulheim (DE); Melanie WIEDEMEIER-JARAD, Dormagen (DE)

(73) Assignee: LANXESS Deutschland GmbH, Cologne (DE)

(21) Appl. No.: 15/413,676

(22) Filed: Jan. 24, 2017

Related U.S. Application Data
(63) Continuation of application No. 14/427,119, filed on Mar. 10, 2015.

(30) Foreign Application Priority Data
Sep. 24, 2012 (EP) 12185724.7
Dec. 21, 2012 (EP) 12198792.9

(51) Int. Cl. C08K 5/375; C07C 319/22; C08K 5/548; C08K 3/36; C08L 9/06; C07C 319/24; C07C 323/62

(52) U.S. Cl. CPC C08K 5/375; C07C 319/24; C07C 319/22; C07C 323/62; C08K 3/36; C08L 9/06; C08K 5/548; C08L 2205/025; C08L 2205/03

(57) ABSTRACT

The present invention relates to polysulfides of the formula (I)

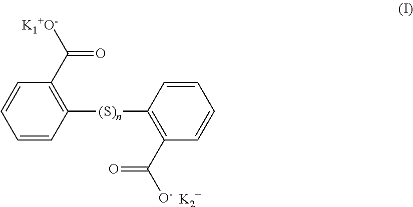

where the cations K₁⁺ and K₂⁺ are each independently any monovalent cation or the fraction of any polyvalent cation which corresponds to a positive charge of one, and n is 2, 3, 4, 5, 6, these having a low residual acidity, and to processes for producing these polysulfide mixtures, to the use of the polysulfides of the formula (I) in rubber mixtures, to the corresponding rubber mixtures, to rubber vulcanizates produced therefrom and to the use thereof.

This application is a continuation of pending U.S. patent application Ser. No. 14/427,119, filed Mar. 10, 2015, with the same title, which claims the right of priority under 35 U.S.C. §119 (a)-(d) and 35 U.S.C. §365 of International Application No. PCT/EP2013/067987, filed Aug. 30, 2013, which is entitled to the right of priority of European Patent Application Nos. 12185724.7 filed Sep. 24, 2012 and 12198792.9, filed Dec. 21, 2012, the contents of which are hereby incorporated by reference in their entirety.”

Silica-containing rubber mixtures are important starting materials, for example for the production of tires with reduced rolling resistance. As they roll, they perform less deformation work (than tires containing only carbon black as filler) and therefore lower fuel consumption. As a result of the obligation to indicate the rolling resistance of tires which has been agreed in some countries, there is a great interest in lowering this resistance further.

To improve the physical properties of the vulcanizates, as mentioned, for example, in DE 2 255 577 A, polysulfidic silanes are used as reinforcing additives. The profile of properties of the rubber vulcanizates thus produced is still not optimal. As well as improvement in the rolling resistance, a particularly desirable property is a low flow viscosity (Mooney viscosity ML 1+4/100° C.) of the rubber mixture, which promotes processibility. For this purpose, further additives have been proposed, such as fatty acid esters, fatty acid salts or mineral oils, which increase flowability but simultaneously reduce stress values under relatively high elongation (e.g. 100% to 300%) or else the hardness of the vulcanizates, such that the reinforcing effect of the filler is not manifested in full. However, too low a hardness or stiffness of the vulcanizate results in unsatisfactory driving characteristics of the tire, particularly on bends. The hardness of the vulcanizate can be increased by increasing the proportion of reinforcing filler or reducing the proportion of plasticizer oil, although each of these two measures causes the disadvantage of higher mixing viscosity in the course of processing.

EP 0 489 313 describes additives which contain glycol functions and have good mechanical properties and improved hysteresis characteristics. Compared to bis[3-(triethoxysilyl)propyl]tetrasulfide according to DE-A 2 255 577, however, the examples do not show any improvement in rolling resistance (tan δ at 60° C.).

EP 1 000 966 achieved an improvement in physical properties through use of a polysulfidic silane in combination with a specific reversion stabilizer in SBR, although no significant change was achieved with respect to the prior art either in terms of mixing viscosity or in terms of rolling resistance (tan δ at 60° C.).

The application EP 11164319, which has not been published to date, describes the use of dibenzoyl tetrasulfide prepared by means of disulfur dichloride together with bis[3-(triethoxysilyl)propyl]tetrasulfide in silica-containing rubber mixtures. Compared to a reference mixture lacking dibenzoyl tetrasulfide, there was only a marginal improvement in the Mooney viscosity; in addition, tensile strength was impaired to a degree, and rolling resistance and abrasion became much worse.

The preparation of 2,2′-tetrathiobisbenzoic acid has been disclosed to date only in U.S. Pat. No. 1,769,423 from 1930. This was obviously done without inert gas, as a result of which a portion of the very oxidation-sensitive mercaptobenzoic acid reacts to give 2,2′-dithiobisbenzoic acid and is then no longer available for the reaction with S₂Cl₂. Moreover, S₂Cl₂ is used in about a 15% excess, as a result of which the product has a significant proportion of chlorine-containing secondary components and a high acidity. Recrystallization from amyl alcohol and further polar solvents, as specified for other products in U.S. Pat. No. 1,769,423, was found in in-house experiments to be unsuitable as for workup of the polysulfides, and led to decomposition of the tetrasulfide, to esterification of the carboxylic acid group or to exchange of active sulfur atoms between the polysulfides and hence to an increase in the proportions of 2,2′-dithiobisbenzoic acid, 2,2′-trithiobisbenzoic acid, 2,2′-pentathiobisbenzoic acid and 2,2′-hexathiobisbenzoic acid, i.e. to a spread in the distribution curve of the product mixture. This is very disadvantageous for use in rubber, since formation of much less active disulfides and trisulfides results in a reduction in the effect of the polysulfides.

Active sulfur atoms are understood to mean sulfur atoms in polysulfides having only sulfur atoms as bonding partners. The exchange of active sulfur atoms does not lead to a change in the elemental composition of a polysulfide mixture, and so cannot be detected by elemental analysis. However, it is possible to detect polysulfide distributions by HPLC, as described by Douglas E. Doster and Melodee Zentner in Journal of Chromatography, 461 (1989) 293-303 and Arisawa Mieko et. al in Tetrahedron Letters, 2005, volume 46, number 28, 4797-4800. This made it clear that the particular polysulfides are present in the form of polysulfide mixtures having a very broad distribution of molecules having different numbers of sulfur atoms (S2, S3, S4, S5, S6).

The problem addressed by the present invention is that of providing novel rubber additives, processes for production thereof and novel rubber mixtures which, in combination with very good flowability of the rubber mixtures, can be converted to vulcanizates with reduced rolling resistance, but in which there is likewise no significant impairment in the likewise important parameters of Shore A hardness, 300 modulus, elongation at break, tensile strength and abrasion.

It has now been found that polysulfides obtained in the reaction of 2-thiobenzoic acid with S₂Cl₂ contain a residual acidity undetectable by simply mixing with water. Thus, a mixture of 10 g of the 2,2′-tetrathiobisbenzoic acid product obtained according to EP 11164319 with 100 ml of water at 25° C. showed a pH of 4.1 and a conductivity of 0.02 mS/cm. Surprisingly, the pH of the aqueous phase after heating the mixture to reflux for 30 minutes and then cooling down to 25° C. was 1.2, and the conductivity was 14 mS/cm.

It has been found that, surprisingly, aftertreatment of the polysulfides obtained in the reaction of 2-thiobenzoic acid with S₂Cl₂ in a nonpolar organic solvent in which the polysulfides preferably have only insignificant solubility, if any, selected from cyclic and/or acyclic hydrocarbon, aromatic hydrocarbon, aliphatic and/or aromatic halohydrocarbon, ether and/or ester solvents that are liquid under reaction conditions, especially toluene, and water at elevated temperature, preferably at a temperature of >60° C., more preferably at a temperature in the range of 80° C.-120° C., now made it possible for the first time to prepare such polysulfides in such a way that they have a distinctly reduced residual acidity and chlorine content, without the aftertreatment resulting in significant broadening of the sulfur distribution of the polysulfides or in the breakdown thereof, such that the resulting polysulfides bring about the improvements in properties required above when used as a rubber additive.

The mixtures of 10 g of the inventive polysulfides and 100 ml of water, after being heated to reflux under standard pressure for 30 minutes and then cooled down to 25° C., have a pH of >2, preferably of 2.5-8, more preferably of 3-7, most preferably of 3.4-6.2. The copied mixtures typically have a conductivity of <5 mS/cm, more preferably <1 mS/cm. In these measurements, the total duration of the cooling phase together with the residence time before the determination of the pH and the conductivity value should not exceed one hour.

In addition, it was found that the above aftertreatment of the polysulfides greatly reduces the chlorine content thereof, which distinctly exceeds 1%. Preferably, the inventive polysulfides have a chlorine content of <1%, preferably <0.3%, more preferably <0.1% and most preferably <0.03%.

The inventive polysulfides consist of compounds of the formula (I)

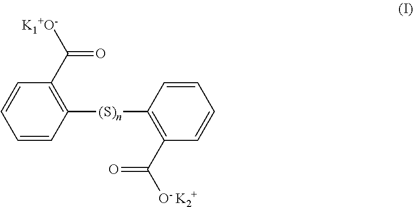

where
K₁⁺ and K₂⁺ are each independently a monovalent cation or the fraction of a polyvalent cation which corresponds to a positive charge of one, and
n is 2, 3, 4, 5 and/or 6.

In a preferred embodiment, K₁⁺ and K₂⁺ are each independently H⁺, an alkali metal cation, especially Li⁺, Na⁺, K⁺, ½ alkaline earth metal cation, especially ½ Mg²⁺, ½ Ca²+, ⅓ Al³⁺, the fraction of a rare earth metal cation which corresponds to a positive charge of one, or ½ Zn²⁺.

Most preferably, K₁⁺ and K₂⁺ are each independently H⁺ or ½ Zn cation, especially ½ Zn cation.

Even though the formal notation in formula (I) could be interpreted to the effect that the compound in which K₁⁺ and/or K₂⁺ is H⁺ is in ionic form as well, the person skilled in the art will appreciate that, in these compounds, a polar covalent O—H bond is present to a predominant degree in place of the ionic bond The present invention preferably provides polysulfide compound(s) of the formula (I) with n=2, 3, 4, 5 and/or 6 in which K₁⁺ and/or K₂⁺ is ½ Zn²⁺. Very particular preference is given to polysulfide compound(s) of the formula (I) with n=4 in which K₁⁺ and/or K₂⁺ is ½ Zn²⁺, especially those in which K₁⁺ and K₂⁺ are ½ Zn²⁺ (cf. formula (II)).

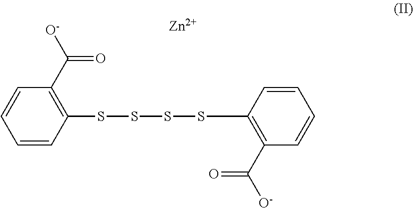 [SMILES: [Zn+2] ; O=C([O-])c1ccccc1SSSSc1ccccc1C(=O)[O-]]

A further preferred embodiment of the invention is that of polysulfide compound(s) of the formula (I) in which K₁⁺ and K₂⁺ are each H⁺ and which therefore correspond to the formula (III) where n=2, 3, 4, 5 and/or 6:

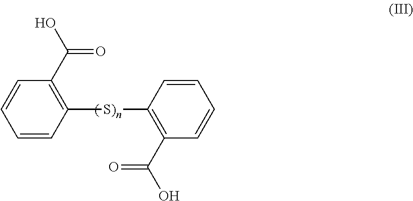

Inventive polysulfides typically consist of a plurality of compounds of the formula (I), in which case, based on the compounds of the formula (I), the cumulative proportion of the compounds of the formula (I) with n=3, n=4, n=5 and n=6 is at least 80%, preferably at least 90%, more preferably at least 95% and most preferably at least 88%. The percentages of the proportions in this invention with regard to the compounds of the formula (I) are always taken directly from the area percentages in the HPLC measurement as specified below in the context of the examples.

In a particularly preferred embodiment, the proportion of the compound of the formula (I) with n=4, based on the compounds of the formula (I), is more than 80%, more preferably more than 90%, even more preferably more than 95%, and most preferably 96%-99%.

Preferably, the proportion of the compound of the formula (I) with n=2, based on the compounds of the formula (I), is less than 10%, more preferably less than 5%, even more preferably less than 2% and most preferably less than 1%.

Polysulfides which are based on compounds of the formula (I) and are preferred in accordance with the invention have a sulfur content according to elemental analysis between 28%-40%, especially between 32%-36%.

Typically, the inventive polysulfides, as a mixture of the compounds of the formula (I), have an average of 3.5-4.5, more preferably 3.7-4.3, even more preferably 3.8-4.2 and most preferably 3.9-4.1 sulfur atoms per molecule of the formula (I).

Preferred polysulfides based on compounds of the formula (I) contain preferably less than 10% by weight, more preferably less than 3% by weight, most preferably less than 1% by weight, of by-products or admixtures, i.e. compounds that do not correspond to the formula (I). More particularly, the content of elemental sulfur and/or sulfur donors based on compounds of the formula (I) is less than 2%, more preferably less than 1%, even more preferably less than 0.3%, most preferably less than 0.1%, and the content of accelerators of the mercapto or sulfenamide group, based on compounds of the formula (I), is less than 2%, more preferably less than 1%, even more preferably less than 0.3% and most preferably less than 0.1%.

The present invention also relates to a process for preparing the inventive polysulfides of the formula (I) by reacting 2-mercaptobenzoic acid with S₂Cl₂ to give compound(s) of the formula (I) where K₁⁺ and K₂⁺ are each H⁺ and n is 2, 3, 4, 5 or 6, preferably 4. Preference is given to conducting the reaction in an inert reaction medium under a protective gas atmosphere at temperatures between 0° C. and 60° C., especially between 15° C. and 35° C. At excessively low temperatures, S₂Cl₂ may not immediately react to completion, which can lead to by-products and to an endangerment potential through the buildup of an elevated S₂Cl₂ concentration in the reaction mixture. Excessively high temperatures should likewise be avoided for reasons of product quality and occupational safety.

Inert reaction media are reaction media which react only insignificantly, if at all, with the reaction components under the reaction conditions, especially aliphatic cyclic and/or acyclic hydrocarbon, aromatic hydrocarbon, aliphatic and/or aromatic halohydrocarbon, ether and ester media that are liquid under reaction conditions. A particularly preferred reaction medium is toluene. Typically, anhydrous or dried reaction media are used in order to avoid side reactions with S₂Cl₂. It is also possible to use mixtures as reaction media. Preference is given to using reaction media and reaction conditions under which the reaction product is dissolved only insignificantly, if at all.

Typically, the reaction medium is initially charged under protective gas, mercaptobenzoic acid is introduced and then S₂Cl₂ is added while cooling the reaction mixture. Mercaptobenzoic acid and S₂Cl₂ can also be introduced simultaneously. Mercaptobenzoic acid can also be initially charged and introduced simultaneously with a reaction medium. Mercaptobenzoic acid and S₂Cl₂ are preferably used in ratios which derive from the stoichiometry, the deviations being <+/−5%, preferably <+/−2%, more preferably <+/−1%. Particular preference is given to avoiding an excess of S₂Cl₂.

Inert gases used in the synthesis of the polysulfides of the formula (I) are preferably noble gases or nitrogen, especially nitrogen. In a preferred embodiment, the HCl gas formed is removed wholly or partly from the mixture as early as during the reaction, especially by passing inert gas, especially nitrogen, through the reaction mixture and/or by applying reduced pressure.

The present process further comprises contacting the resulting polysulfides of the formula (I) with water or a mixture of water and an inert organic medium in which the polysulfides preferably have only insignificant solubility, if any, especially in cyclic and/or acyclic hydrocarbon, aromatic hydrocarbon, aliphatic and/or aromatic halohydrocarbon, ether and ester media that are liquid under reaction conditions, more preferably toluene, and heating to a temperature of >60° C., preferably to 80° C.-120° C. The duration of the heating is preferably more than 30 minutes, more preferably 1 h-10 h, even more preferably 2 h-6 h. Preferably, the reaction with S₂Cl₂ with mercaptobenzoic acid and the subsequent treatment with water are conducted without intermediate isolation, especially in a one-pot process. In a preferred embodiment, the reaction suspension, after water has been fed in, is heated to reflux and/or a portion of the reaction medium is distilled off as an azeotrope with water.

In an alternative embodiment, in place of or after the above treatment with water, the polysulfides of the formula (I) in which K₁⁺ and/or K₂⁺ is H⁺ are contacted with salts of metals containing cations K₁⁺ and/or K₂⁺ and heated, preferably in aqueous dispersion, to a temperature of >60° C., preferably to 80° C.-120° C., which affords inventive polysulfides of the formula (I) in which at least one of the cations K₁⁺ and/or K₂⁺ is a cation other than H⁺. The duration of the heating is preferably more than 30 minutes, more preferably 1 h-10 h, even more preferably 2 h-6 h. The pH in the preparation of the polysulfides of the formula (I) in which at least one of the cations K₁⁺ and/or K₂⁺ is a cation other than H⁺ is preferably in the range of 2-8, especially in the range of 3-7. Metal salt solutions used are preferably sulfates, hydrogensulfates, phosphates, hydrogenphosphates, dihydrogenphosphates, carbonates, hydrogencarbonates, hydroxides, nitrates, chlorides and acetates, especially sulfates. Alkali metal hydroxides, especially alkali metal hydroxide solutions, are preferably used as auxiliary bases in order to set an optimal reaction pH range. The compounds of the formula (I) obtained by reaction with the metal salts, especially compounds of the formula (I) in which K₁⁺ and/or K₂⁺ is ½ Zn²⁺, do not just have the pH required after heating 10 g of polysulfides in 100 ml of water to reflux for 30 min and then cooling down to 25° C. but surprisingly also show a distinct improvement in the profile of properties of vulcanized rubber mixtures, like the inventive compounds of the formula (I) in which K₁⁺ and/or K₂⁺ is H⁺.

The process according to the invention can be conducted in batchwise mode, in continuous mode or in cascade mode.

The process according to the invention affords compounds of the formula (I) which, on mixing of 10 g of the compounds of the formula (I) with 100 ml of water, after heating to reflux for 30 minutes and subsequent cooling to 25° C., have a pH of >2, preferably of 2.5 to 8, more preferably of 3-7, very particularly of 3.4-6.2. The cooled mixtures typically have a conductivity of <5 mS/cm, more preferably <1.

The chlorine content of the inventive polysulfides is <1%, preferably <0.3%, more preferably <0.1% and even more preferably <0.03%.

The process according to the invention additionally enables preparation of polysulfides based on compounds of the formula (I) having a very small sulfur distribution range, i.e. a very high proportion of compounds of the formula (I) with n=4 and a very low proportion of compounds of the formula (I) with n=2, especially with a very low proportion of compounds of the formula (I) with n=2, 3, 5 and 6.

Through the process according to the invention involving contacting the resulting polysulfides of the formula (I) with water or a mixture of water and an inert organic medium in which the polysulfides preferably have only insignificant solubility, if any, especially in cyclic and/or acyclic hydrocarbon, aromatic hydrocarbon, aliphatic and/or aromatic halohydrocarbon, ether and ester media that are liquid under reaction conditions, more preferably toluene, and heating to a temperature of >60° C., preferably to 80° C.-120° C., it is possible to obtain polysulfides of the formula (I) in which K₁⁺ and K₂⁺ are each H⁺ and in which the proportion of the compound with n=4, based on the compounds of the formula (I), is more than 80%, more preferably more than 90%, even more preferably more than 95% and most preferably 96%-99%. These polysulfides feature a melting range which ends at at least 300° C., preferably at least 302° C. and even more preferably at least 304° C. The end of the melting range can be visually determined exactly with the Büchi Melting Point B-545 melting point apparatus. The heating rate is 1° C./min, starting from a temperature of 290° C. In addition, these polysulfides are in a novel crystal polymorph, called “beta crystal polymorph” hereinafter for better distinguishability, as opposed to the polysulfides obtained directly from the reaction mixture, called “alpha crystal polymorph” hereinafter. The beta crystal polymorph shows a dominant signal in the x-ray diffractogram (Cu K-alpha radiation) at a diffraction angle 2theta (°) of 27.2 and further intense signals at diffraction angles 2theta (°) of 21.0 and 13.5, whereas the alpha crystal polymorph exhibits a dominant signal at a diffraction angle 2theta (°) of 26.6 and further intense signals at diffraction angles 2theta (°) of 21.1 and 14.6. The statement of the diffraction angle 2theta (°) is subject to the standard range of fluctuation of +/−0.1. The present invention preferably provides polysulfides of the beta crystal polymorph having a dominant signal at a diffraction angle 2theta (°) of 27.2. Surprisingly, it was also possible to eliminate the significant odor nuisance which is customary for these compounds, since the polysulfides obtained have only a weak intrinsic odor.

Preferably, the inventive polysulfides of the formula (I), after preparation, are stored at temperatures between 0-35° C.

It has now been found that, surprisingly, the inventive polysulfides of the formula (I) improve the flowability of rubber mixtures and afford vulcanizates having a good profile of properties and especially a low rolling resistance.

The present invention further provides polysulfides of the formula (I) obtainable by reacting 2-mercaptobenzoic acid with S₂Cl₂ to give compound(s) of the formula (I) where K₁⁺ and K₂⁺ are each H⁺ and n is 2, 3, 4, 5 or 6, preferably 4, preferably by reaction in an inert reaction medium under a protective gas atmosphere at temperatures between 0° C. and 60° C., especially between 15° C. and 35° C., and aftertreatment by suspension in an inert organic medium in which the polysulfides preferably have only insignificant solubility, if any, selected from cyclic and/or acyclic hydrocarbon, aromatic hydrocarbon, aliphatic and aromatic halohydrocarbon, ether and ester media that are liquid under reaction conditions, especially toluene, and contacting with water at temperatures of >60° C., especially 80° C.-120° C. Most preferably, the reaction suspension, after water has been fed in, is heated to reflux and/or a portion of the reaction medium is distilled off, especially as an azeotrope with water. Most preferably, the polysulfides of the formula (I) have a proportion of the compound with n=4, based on the total amount of the compounds of the formula (I), of more than 80%, more preferably more than 90%, even more preferably more than 95% and most preferably 96%-99%, and are in the beta crystal polymorph.

The invention therefore further provides rubber mixtures each comprising at least a rubber and the inventive polysulfides of the formula (I).

The invention especially provides rubber mixtures each comprising at least a rubber, a sulfur-containing alkoxysilane, a silica-based filler and the inventive compounds of the formula (I).

The inventive polysulfides of the formula (I) may also be used partly or fully in absorbed form on inert organic or inorganic supports. Preferred support materials are silica, natural and synthetic silicates, alumina and/or carbon blacks.

The total content of the inventive polysulfides of the formula (I) in preferred rubber mixtures is 0.1 to 15 phr, more preferably 0.3 to 7 phr, even more preferably 0.5 to 3 phr and most preferably 0.7 to 1.5 phr. The unit phr stands for parts by weight based on 100 parts by weight of rubber used in the rubber mixture.

For the production of the inventive rubber mixtures, it is possible to use natured rubber and/or synthetic rubbers. Preferred synthetic rubbers are, for example,
BR—polybutadiene
ABR—butadiene/C₁-C₄-alkyl acrylate copolymers
CR—polychloroprene
IR—polyisoprene
SBR—styrene/butadiene copolymers having styrene contents of 1%-60%, preferably 20%-50%, by weight
IIR—isobutylene/isoprene copolymers
NBR—butadiene/acrylonitrile copolymers having acrylonitrile contents of 5%-60%, preferably 10%-50%, by weight
HNBR—partly hydrogenated or fully hydrogenated NBA rubber
EPDM—ethylene/propylene/diene copolymers
   and mixtures of two or more of these rubbers.

Preferably, the inventive rubber mixtures comprise at least one SBR rubber and at least one BR rubber, more preferably in a weight ratio of SBR:BR of 60:40 to 90:10.

In an advantageous embodiment, the inventive rubber mixtures additionally comprise at least one NR rubber. More preferably, they include at least one SBR rubber, at least one BR rubber and at least one NR rubber, the weight ratio of SBR rubber to BR rubber to NR rubber most preferably being 60 to 85:10 to 35:5 to 20.

Suitable sulfur-containing alkoxysilanes for the inventive rubber mixtures are, for example, bis(triethoxysilylpropyl)tetrasulfane (e.g. Si 69 from Evonik), bis(triethoxysilylpropyl)disulfane (e.g. Si 75 from Evonik), 3-(triethoxysilyl)-1-propanethiol, polyether-functionalized mercaptosilanes such as Si 363 from Evonik, thioester-functionalized alkoxysilanes such as NXT or NXT Z from Momentive (formerly GE). It is also possible to use mixtures of the sulfur-containing alkoxysilanes. Liquid sulfur-containing alkoxysilanes may be applied to a support for better meterability and/or dispersibility (dry liquid). The active ingredient content is between 30 and 70 parts by weight, preferably 40 and 60 parts by weight, per 100 parts by weight of dry liquid.

The proportion of the sulfur-containing alkoxysilanes in the inventive rubber mixtures is preferably 2 to 20 phr, more preferably 3 to 11 phr and most preferably 5 to 8 phr, calculated in each case as 100% active ingredient. Preferably, the amount of sulfur-containing alkoxysilane is greater than or equal to the amount of inventive polysulfides of the formula (I). More preferably, the weight ratio of sulfur-containing alkoxysilane to the inventive polysulfides of the formula (I) is 1.5:1 to 20:1, even more preferably 3:1 to 15:1 and most preferably 5:1 to 10:1.

The rubber mixture preferred in accordance with the invention additionally comprises one or more silica-based fillers. Preference is given to using the following substances for this purpose:
silica, especially precipitated silica or fumed silica, produced, for example, by precipitation of solutions of silicates or flame hydrolysis of silicon halides having specific surface areas of 5-1000, preferably 20-400, m²/g (BET surface area) and having primary particle sizes of 10-400 nm. The silicas may optionally also be present as mixed oxides with other metal oxides, such as oxides of Al, Mg, Ca, Ba, Zn, Zr, Ti,
synthetic silicates, such as aluminum silicate, alkaline earth metal silicates such as magnesium silicate or calcium silicate, having BET surface areas of 20-400 m²/g, preferably of 100-400 m²/g, and primary particle size of 10-400 nm,
natural silicates, such as kaolin and other natural occurring silicas,
glass fibers, including in the forms of mats and strands,
glass microbeads.

It is of course possible to use additional fillers. Especially suitable for this purpose are carbon blacks produced by the thermal black, furnace black or gas black process, having BET surface areas of 20-200 m²/g, such as SAF, ISAF, IISAF, HAF, FEF or GPF carbon blacks.

The total content of fillers is preferably 10 to 200 phr, more preferably 50 to 160 phr and most preferably 60 to 120 phr. The proportion by weight of silica-based fillers is at least 10%, preferably at least 20%, more preferably at least 50% and most preferably at least 80% of the total filler content.

A particularly preferred embodiment is the combination of silica, carbon black and inventive polysulfides of the formula (I). In this case, the ratio of silica to carbon black may vary within arbitrary limits, preference being given to a silica:carbon black weight ratio of 20:1 to 1.5:1 for use in tires.

In a preferred embodiment, the inventive rubber mixtures also comprise one or more crosslinkers. For this purpose, sulfur-based or peroxidic crosslinkers in particular are suitable, particular preference being given to sulfur-based crosslinkers.

Peroxidic crosslinkers used are preferably bis(2,4-dichlorobenzyl) peroxide, dibenzoyl peroxide, bis(4-chlorobenzoyl) peroxide, 1,1-bis(t-butylperoxy)-3,3,5-trimethylcyclohexane, tert-butyl perbenzoate, 2,2-bis(t-butylperoxy)butane, 4,4-di-tert-butyl peroxynonylvalerate, dicumyl peroxide, 2,5-dimethyl-2,5-di(tert-butylperoxy)hexane, tert-butyl cumyl peroxide, 1,3-bis(t-butylperoxyisopropyl)benzene, di-tert-butyl peroxide and 2,5-dimethyl-2,5-di(tert-butylperoxy)-3-hexyne.

It may be advantageous to use, as well as these peroxidic crosslinkers, also further additions which can help to increase the crosslinking yield: Suitable examples for this purpose are triallyl isocyanurate, triallyl cyanurate, trimethylolpropane tri(meth)acrylate, triallyl trimellitate, ethylene glycol di(meth)acrylate, butanediol di(meth)acrylate, trimethylolpropane tri(meth)acrylate, zinc diacrylate, zinc dimethacrylate, 1,2-polybutadiene or N,N′-m-phenylenedimaleimide.

Crosslinkers used may be sulfur in elemental soluble or insoluble form or in the form of sulfur donors. Useful sulfur donors include, for example, dimorpholyl disulfide (DTDM), 2-morpholinodithiobenzothiazole (MBSS), caprolactam disulfide, dipentamethylenethiuram tetrasulfide (DPTT) and tetramethylthiuram disulfide (TMTD).

In principle, the crosslinking of the inventive rubber mixtures can be effected with sulfur or sulfur donors alone, or together with vulcanization accelerators, suitable examples of which are, for example, dithiocarbamates, thiurams, thiazols, sulfenamides, xanthogenates, bi- or polycyclic amines, guanidine derivatives, dithiophosphates, caprolactams and thiourea derivatives. In addition, zinc diaminediisocyanate, hexamethylenetetramine, 1,3-bis(citraconimidomethyl)benzene and cyclic disulfanes are also suitable. Preferably, the inventive rubber mixtures comprise sulfur-based crosslinkers and vulcanization accelerators.

Particular preference is given to using sulfer, magnesium oxide and/or zinc oxide as crosslinking agents, to which the known vulcanization accelerators are added, such as mercaptobenzothiazoles, thiazolesulfenamides, thiurams, thiocarbamates, guanidines, xanthogenates and thiophosphates.

The crosslinking agents and vulcanization accelerators are preferably used in amounts of 0.1 to 10 phr, more preferably of 0.1 to 5 phr.

The inventive rubber mixtures may comprise further rubber auxiliaries such as reaction accelerators, aging stabilizers, thermal stabilizers, light stabilizers, antioxidants, especially antiozonants, flame retardants, processing auxiliaries, impact modifiers, plasticizers, tackifiers, blowing agents, dyes, pigments, waxes, extenders, organic acids, retardants, metal oxides and activators, especially triethanolamine, polyethylene glycol, hexanetriol and reversion stabilizers.

These rubber auxiliaries are used in customary amounts directed by factors including the end use of the vulcanizates. Typical amounts are 0.1 to 30 phr.

Aging stabilizers used are preferably alkylated phenols, styrenized phenol, sterically hindered phenols such as 2,6-di-tert-butylphenol, 2,6-di-tert-butyl-p-cresol (BHT), 2,6-di-tert-butyl-4-ethylphenol, sterically hindered phenols containing ester groups, thioether-containing sterically hindered phenols, 2,2′-methylenebis(4-methyl-6-tert-butylphenol) (BPH) and sterically hindered thiobisphenols.

If discoloration of the rubber is unimportant, it is also possible to use aminic aging stabilizers, for example mixtures of diaryl-p-phenylenediamines (DTPD), octylated diphenylamine (ODPA), phenyl-α-naphthylamine (PAN), phenyl-β-naphthylamine (PBN), preferably those based on phenylenediamine, for example N-isopropyl-N′-phenyl-p-phenylenediamine, N-1,3-dimethylbutyl-N′-phenyl-p-phenylenediamine (6PPD), N-1,4-dimethylpentyl-N′-phenyl-p-phenylenediamine (7PPD), N,N′-bis(1,4-dimethylpentyl)-p-phenylenediamine (77PD).

Further aging stabilizers are phosphites such as tris(nonylphenyl) phosphite, polymerized 2,2,4-trimethyl-1,2-dihydroquinoline (TMQ), 2-mercaptobenzimidazole (MBI), methyl-2-mercaptobenzimidazole (MMBI), zinc methylmercaptobenzimidazole (ZMMBI), which are usually used in combination with the above phenolic aging stabilizers. TMQ, MBI and MMBI are used in particular for NBR rubbers which are vulcanized using peroxides.

Ozone resistance can be improved by means of antioxidants, for example N-1,3-dimethylbutyl-N′-phenyl-p-phenylenediamine (6PPD), N-1,4-dimethylpentyl-N′-phenyl-p-phenylenediamine (7PPD), N,N′-bis(1,4-dimethylpentyl)-p-phenylenediamine (77PD), enol ethers or cyclic acetals.

Processing auxiliaries should be active between the rubber particles and should counter frictional forces in the course of mixing, plasticizing and forming. Processing auxiliaries which may be present in the inventive robber mixtures include all the lubricants customary for the processing of plastics, for example hydrocarbons such as oils, paraffins and PE waxes, fatty alcohols having 6 to 20 carbon atoms, ketones, carboxylic acids such as fatty acids and montanic acids, oxidized PE wax, metal salts of carboxylic acids, carboxamides and carboxylic esters, for example with the alcohols ethanol, fatty alcohols, glycerol, ethanediol, pentaerythritol and long-chain carboxylic acids as the acid component.

In order to reduce flammability and to decrease evolution of smoke in the event of burning, the inventive rubber mixture composition may also comprise flame retardants. For this purpose, for example, antimony trioxide, phosphoric esters, chloroparaffin, aluminum hydroxide, boron compounds, zinc compounds, molybdenum trioxide, ferrocene, calcium carbonate or magnesium carbonate are used.

Prior to crosslinking, further plastics may also be added to the rubber vulcanizate, these acting, for example, as polymeric processing auxiliaries or impact modifiers. These plastics are preferably selected from the group consisting of the homo- and copolymers based on ethylene, propylene, butadiene, styrene, vinyl acetate, vinyl chloride, glycidyl acrylate, glycidyl methacrylate, acrylates and methacrylates having alcohol components of branched or unbranched C₁ to C₁₀ alcohols, particular preference being given to polyacrylates having identical or different alcohol residues from the group of the C₄ to C₈ alcohols, especially of butanol, hexanol, octanol and 2-ethylhexanol, polymethylmethacrylate, methyl methacrylate-butyl acrylate copolymers, methyl methacrylate-butyl methacrylate copolymers, ethylene-vinyl acetate copolymers, chlorinated polyethylene, ethylene-propylene copolymers, ethylene-propylene-diene copolymers.

In a preferred embodiment, the inventive rubber mixture contains 0.1 to 15 phr of the reversion stabilizer 1,6-bis(N,N-dibenzylthiocarbamoyldithio)hexane (CAS no.: 151900-44-6), which enables a decrease in tan δ (60° C.), i.e. in the rolling resistance, improves abrasion values and shortens scorch time and vulcanization time.

Preferably, it is a feature of the inventive rubber mixtures that a vulcanizate cured therefrom at 170° C./t95 has a loss factor tan δ at 60° C. of <0.16, more preferably <0.12, especially <0.10, and at the same time a Shore A hardness at 23° C. of >66. In combination therewith, the inventive rubber mixtures can also achieve a vulcanization time of less than 2000 seconds and a 300 modulus value of >15 MPa.

The present invention further provides a process for producing rubber mixtures by mixing at least one rubber with at least one silica-based filler, a sulfur-containing alkoxysilane and at least one inventive polysulfide mixture. This preferably involves using 10 to 150 phr, more preferably 30 to 120 phr and most preferably 50 to 100 phr of filler, 0.1 to 15 phr, more preferably 0.3 to 7 phr, even more preferably 0.5 to 3 phr and most preferably 0.7 to 1.5 phr of inventive polysulfides of the formula (I), and 2 to 20 phr, more preferably 3-11 phr and most preferably 5 to 8 phr of the sulfur-containing alkoxysilane. In addition, in the mixing process, the abovementioned additional fillers, crosslinkers, vulcanization accelerators and rubber auxiliaries may be added, preferably in the amounts specified above.

In the multistage mixing process, the inventive polysulfides of the formula (I) are preferably added in the first part of the mixing process, and one or more crosslinkers, especially sulfur, and optionally vulcanization accelerators in a later mixing stage. The temperature of the rubber composition is preferably 100 to 200° C., more preferably 120° C. to 170° C. The shear rates in the course of mixing are 1 to 1000 sec⁻¹, preferably 1 to 100 sec⁻¹. In a preferred embodiment, the rubber mixture is cooled after the first mixing stage and the crosslinker and optionally crosslinking accelerator and/or additives which help to increase the crosslinking yield are added in a later mixing stage at <140° C., preferably <100° C. It is likewise possible to add the inventive polysulfides of the formula (I) in a later mixing stage and at lower temperatures such as 40 to 100° C., for example together with sulfur and crosslinking accelerator.

The blends of the rubber with the filler and the inventive polysulfides of the formula (I) can be conducted in customary mixing units such as rollers, internal mixers and mixing extruders.

The optional addition of 1,6-bis(N,N-dibenzylthiocarbamoyldithio)hexane preferably takes place in the first stage of the multistage mixing process.

The present invention further provides a process for vulcanizing the inventive rubber mixtures, which is preferably conducted at blend temperatures of 100 to 200° C., more preferably at 130 to 180° C. In a preferred embodiment, the vulcanization takes place at a pressure of 10 to 200 bar.

The present invention also comprises rubber vulcanizates obtainable by vulcanizing the inventive rubber mixtures. These vulcanizates, especially when used in tires, have the advantages of an excellent profile of properties and an unexpectedly low rolling resistance.

The inventive rubber vulcanizates are suitable for production of moldings having improved properties, for example for the production of cable sheaths, hoses, drive belts, conveyor belts, roll coverings, tires, shoe soles, gasket rings and damping elements.

The inventive rubber vulcanizate can additionally be used for production of foams. For this purpose, chemical or physical blowing agents are added thereto. Useful chemical blowing agents include all the substances known for this purpose, for example azodicarbonamide, p-toluenesulfonyl hydrazide, 4,4′-oxybis(benzenesulfohydrazide), p-toluenesulfonyl semicarbazide, 5-phenyltetrazole, N,N′-dinitrosopentamethylenetetramine, zinc carbonate or sodium hydrogencarbonate, and mixtures comprising these substances. Examples of suitable physical blowing agents are carbon dioxide or halohydrocarbons.

The present invention further provides for the use of the inventive polysulfides of the formula (I) for production of rubber mixtures and vulcanizates thereof. Preferably, each of the rubber mixtures comprises at least a rubber, a filler and the inventive polysulfides of the formula (I), more preferably at least a rubber, a sulfur-containing alkoxysilane, a silica-based filler and the inventive polysulfides of the formula (I).

It has been found that, surprisingly, mixtures comprising at least one sulfur-containing alkoxysilane, especially bis(triethoxysilylpropyl)tetrasulfane, bis(triethoxysilylpropyl)disulfane, 3-(triethoxysilyl)-1-propanethiol, polyether-functionalized mercaptosilane or thioester-functionalized alkoxysilane and inventive polysulfides of the formula (I), in spite of the reactive groups, have adequate compatibility of the components, which enables homogeneous introduction into rubber mixtures and exact dosage in the desired ratio.

Therefore, the present invention also encompasses mixtures usable as an additive, and the use of sulfur-containing alkoxysilanes, especially bis(triethoxysilylpropyl)tetrasulfane, bis(triethoxysilylpropyl)disulfane, 3-(triethoxysilyl)-1-propanethiol, polyether-functionalized mercaptosilanes or thioester-functionalized alkoxysilanes, and inventive polysulfides of the formula (I) for production of these mixtures. Preferably, the weight ratio of alkoxysilane, especially of bis(triethoxysilylpropyl)tetrasulfane and/or of bis(triethoxysilylpropyl)disulfane, to inventive polysulfides of the formula (I) is 1.5:1 to 20:1, more preferably 3:1 to 15:1 and most preferably 5:1 to 10:1.

### Determination of the Properties of the Rubber Mixture or Vulcanizates

### Mooney Viscosity Measurement:

The viscosity can be determined directly from the force with which the rubbers (and rubber mixtures) resist processing. In the Mooney shearing disk viscometer, a grooved disk surrounded by sample substance above and below is turned at about two revolutions per minute in a heatable chamber. The force required therefor is measured as the torque and corresponds to the respective viscosity. The sample is generally preheated to 100° C. for one minute; the measurement takes 4 minutes more, for which the temperature is kept constant. The viscosity is reported together with the particular test conditions, for example ML (1+4) 100° C. (Mooney viscosity, large rotor, preheating time and test time in minutes, test temperature).

### Scorch Characteristics (Scorch Time t5):

In addition, the same test as described above can also be used to measure the scorch characteristics of a mixture. The temperature chosen was 130° C. The rotor runs until the torque value, after passing through a minimum, has risen to 5 Mooney units relative to the minimum value (t5). The greater the value (unit: seconds), the slower the incidence of scorch. In practice, a scorch time of more than 300 seconds is usually advantageous.

### Rheometer (Vulcameter) Vulcanization Time 170° C./t95:

The MDR (moving die rheometer) vulcanization profile and the associated analytical data are measured on a Monsanto MDR 2000 rheometer in accordance with ASTM D5289-95. The vulcanization time is determined as the time at which 95% of the rubber has been crosslinked. The temperature chosen was 170° C.

### Determination of Hardness:

To determine the hardness of the inventive rubber mixture, milled sheets of thickness 6 mm were produced from the rubber mixture according to the formulations in table 1. Specimens of diameter 35 mm were cut out of the milled sheets, and the Shore A hardness therefore was determined by means of a digital Shore hardness tester (Zwick GmbH & Co. KG, Ulm). The hardness of a rubber vulcanizate gives a first indication of its stiffness.

### Tensile Test:

The tensile test serves to directly determine the load limits of an elastomer and is effected to DIN 53504. The increase in length on fracture is based on the starting length and corresponds to the elongation at break. In addition, the force on attainment of particular elongation stages, usually 50%, 100%, 200% and 300%, is also determined and expressed as the strain value (tensile strength at the specified elongation of 300%, or 300 modulus).

### Dynamic Damping:

Dynamic test methods are used to characterize the deformation characteristics of elastomers under periodically altered loads. A stress applied on the outside changes the conformation of the polymer chain. The loss factor tan δ is determined indirectly via the ratio between loss modulus G″ and storage modulus G′. The loss factor tan δ at 60° C. is proportional to the rolling resistance and should be at a minimum.

### Abrasion:

The abrasion gives an indication of wear and hence the lifetime of a product. Abrasion was determined to DIN 53516. For economic and ecological reasons, the aim is a low value.

### EXAMPLE 1

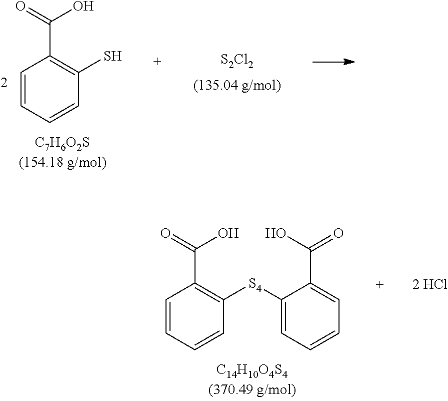

Apparatus: 2000 ml four-neck flask with thermometer, dropping funnel with pressure equalizer, reflux condenser with gas outlet attachment (bubble counter) and hose, stirrer, gas inlet tube

Initial charge: 118.0 g=0.75 mol of mercaptobenzoic acid (Merck, ≧98%), 900 ml of toluene (p.A., from Merck, dried over molecular sieve)

Feed: 51.15 g=0.375 mol of disulfur dichloride (≧99%, from Merck)

The nitrogen-purged apparatus is initially charged with dried toluene and mercaptobenzoic acid. The disulfur dichloride is then added dropwise to the suspension present under a nitrogen flow at a temperature of 0-5° C. within about 1 h. The metering rate should be adjusted such that a temperature of 5° C. is not exceeded. After the reaction has ended, stirring of the mixture continues under a nitrogen flow at room temperature overnight. Subsequently, the reaction solution is filtered with suction through a D4 frit and washed through 2× with about 200 ml of dried toluene. The product is dried at room temperature (about 25° C.) in a vacuum drying cabinet.

Yield: 144.6 g (104.1%) of a polysulfide mixture of the idealized formula

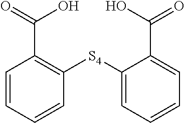

Elemental analysis: C: 45.0% H: 3.0% O: 16.7% S: 33.6% Cl: 1.4%

The melting range and the end of the melting range were determined visually with the Büchi Melting Point B-545 melting point apparatus. The heating rate was 1° C./min, starting from a temperature of 290° C.

| | |
|---|---|
| Melting range: 296-299° C. | Melting range (end): 299° C. |

An x-ray diffraction analysis was conducted.

| | |
|---|---|
| Diffractometer: | PANalytical X'Pert PRO |
| Geometry: | transmission |
| Primary monochromator | focusing x-ray mirror |
| Detector: | PixCEL 2D |
| Radiation: | Cu K-alpha |
| Tube voltage: | 40 kV |
| Tube current: | 40 mA |
| Measurement range: | 2-80° in 2theta |
| Step width in 2theta: | 0.013° |
| Step time: | 100 s |
| Evaluation software: | PANalytical HighScorePlus v.3 |

The x-ray diffractogram (Cu K-alpha radiation) shows the following three largest signals:

| | |
|---|---|
| Diffraction angle 2theta (°): 26.6 | Relative intensity: 100% |
| Diffraction angle 2theta (°): 14.6 | Relative intensity: 75% |
| Diffraction angle 2theta (°): 21.1 | Relative intensity: 54% |

The x-ray diffractogram (Cu K-alpha radiation) does not show any signal at a diffraction angle 2theta (°) of 27.2.

It is obvious from the dominant signal at a diffraction angle 2theta (°) of 26.6 that the product is of the alpha crystal polymorph.

The product has a very intense odor.

The mixture of 10 g of polysulfide mixture with 100 ml of water, after being refluxed for 30 min and cooled down to 25° C., exhibited a pH of 1.2 and a conductivity of 14 mS/cm.

### EXAMPLE 2

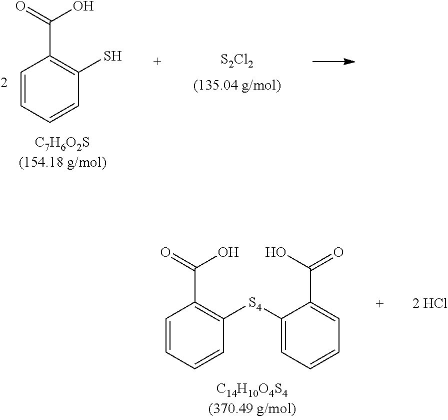

Apparatus: 2000 ml four-neck flask with thermometer, dropping funnel with pressure equalizer, reflux condenser with gas outlet attachment (bubble counter) and hose, stirrer, gas inlet tube

Initial charge: 118.0 g=0.75 mol of mercaptobenzoic acid (Merck, ≧98%), 900 ml of toluene (p.A., from Merck, dried over molecular sieve)

Feed: 51.15 g=0.375 mol of disulfur dichloride (≧99%, from Merck)

The nitrogen-purged apparatus is initially charged with dried toluene and mercaptobenzoic acid. The disulfur dichloride is then added dropwise to the suspension present under a nitrogen flow at a temperature of 20-25° C. within about 30 min. The metering rate should be adjusted such that a temperature of 25° C. is not exceeded. After the reaction has ended, stirring of the mixture continues under a nitrogen flow at 20-25° C. for 1 h. Then 20 ml of demineralized water and 180 ml of toluene are added and the mixture is heated to reflux under a nitrogen flow for 2 h. Subsequently, about 200 ml are distilled off under standard pressure with a nitrogen flow.

The mixture is cooled to room temperature. Subsequently, the reaction solution is filtered with suction through a D4 frit and washed through 2× with about 200 ml of dried toluene. The product is dried at room temperature (about 25° C.) in a vacuum drying cabinet.

Yield: 139.5 g (100.4%) of a polysulfide mixture of the idealized formula

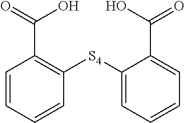

Elemental analysis: C: 45.2% H: 2.7% O: 18.1% S: 33.6% Cl: 0.05%

The melting range and the end of the melting range were determined visually with the Büchi Melting Point B-545 melting point apparatus. The heating rate was 1° C./min, starting from a temperature of 290° C.

| | |
|---|---|
| Melting range: 302-305° C. | Melting range (end): 305° C. |

The x-ray diffractogram (Cu K-alpha radiation) shows the following three largest signals:

| | |
|---|---|
| Diffraction angle 2theta (°): 27.2 | Relative intensity: 100% |
| Diffraction angle 2theta (°): 21.0 | Relative intensity: 30% |
| Diffraction angle 2thela (°): 13.5 | Relative intensity: 24% |

It is obvious from the dominant signal at a diffraction angle 2theta (°) of 27.2 that the product is of the beta crystal polymorph.

The product was analyzed by RP-HPLC and time-of-flight mass spectrometry (TOF MS).

The percentages with regard to the compounds of the formula (I) with K₁⁺ and K₂⁺=H⁺ are taken directly from the area percentages of HPLC measurement with a UV detector:

<1% compound with n=2, 1% compound with n=3, 97% compound with n=4, 1% compound with n=5, <1% compound with n=6.

HPLC instrument: Agilent 1100 Series with degasser, binary pump, column oven, variable wavelength detector and autosampler

| | |
|---|---|
| Stationary phase: | Inertsil ODS-3, particle diameter 3 μm |
| Column length: | 150 mm |
| Internal diameter of column: | 2.1 mm |
| Mobile phase | A: 100% water + 25 mmol |
| | ammonium acetate |
| | B: 95% methanol: 5% water + |
| | 25 mmol ammonium acetate |

Elution program:

| Time (min) | Eluent A (% by vol.) | Eluent B (% by vol.) |
|---|---|---|
| 0 | 80 | 20 |
| 5 | 80 | 20 |
| 30 | 1 | 99 |
| 60 | 1 | 99 |
| 62 | 80 | 20 |

| | | |
|---|---|---|
| Column temperature: | 40° | C. |
| Flow rate: | 0.3 | ml/min |
| Run time: | 72 | min |
| Injection volume: | 5 | μl |
| Wavelength of UV detector: | 225 | nm |

A 50 mg sample of product to be analyzed was weighed into a 50 ml volumetric flask, dissolved by adding about 10 ml of tetrahydrofuran and making the mixture up to the calibration mark with tetrahydrofuran.

The product has a slight intrinsic odor.

The mixture of 10 g of polysulfide mixture with 100 ml of water, after being refluxed for 30 min and cooled down to 25° C., exhibited a pH of 3.4 and a conductivity of 0.2 mS/cm.

### EXAMPLE 3

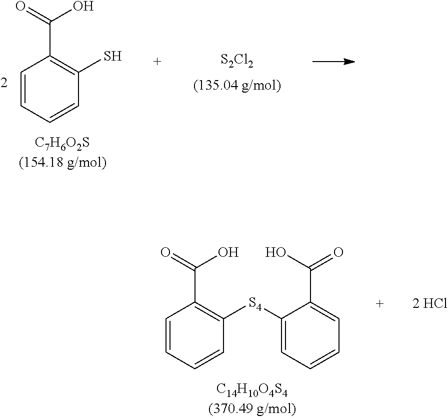

Apparatus: 2000 ml four-neck flask with thermometer, dropping funnel with pressure equalizer, reflux condenser with gas outlet attachment (bubble counter) and hose, stirrer, gas inlet tube

Initial charge: 236.0 g=1.5 mol of mercaptobenzoic acid (Merck, ≧98%), 1000 ml of toluene (p.A., from Merck, dried over molecular sieve)

Feed: 102.3 g=0.75 mol of disulfur dichloride (≧99%, from Merck)

The nitrogen-purged apparatus is initially charged with dried toluene and mercaptobenzoic acid. The disulfur dichloride is then added dropwise to the suspension present under a nitrogen flow at a temperature of 20-25° C. within about 30 min. The metering rate should be adjusted such that a temperature of 25° C. is not exceeded. After the reaction has ended, stirring of the mixture continues under a nitrogen flow at 20-25° C. for 1 h. Then 100 ml of demineralized water are added and the mixture is heated to reflux under a nitrogen flow for 4 h.

The mixture is cooled to room temperature. Subsequently, the reaction solution is filtered with suction through a D4 frit and washed through 2× with about 300 ml of toluene. The product is dried at about 50° C. in a vacuum drying cabinet.

Yield: 279 g (100.4%) of a polysulfide mixture of the idealized formula

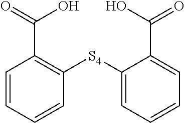

Elemental analysis: C: 45.3% H: 2.9% O: 17.4% S: 34.5% Cl: 0.02%

The melting range and the end of the melting range were determined visually with the Büchi Melting Point B-545 melting point apparatus. The heating rate was 1° C./min, starting from a temperature of 290° C.

| | |
|---|---|
| Melting range: 302-305° C. | Melting range (end): 305° C. |

The x-ray diffractogram (Cu K-alpha radiation) shows the following three largest signals:

| | |
|---|---|
| Diffraction angle 2theta (°): 27.2 | Relative intensity: 100% |
| Diffraction angle 2theta (°): 21.0 | Relative intensity: 22% |
| Diffraction angle 2theta (°): 13.5 | Relative intensify: 21% |

It is obvious from the dominant signal at a diffraction angle 2theta (°) of 27.2 that the product is of the beta crystal polymorph.

The product was analyzed by RP-HPLC and time-of-flight mass spectrometry (TOF MS).

The percentages with regard to the compounds of the formula (I) with K₁⁺ and K₂⁺=H⁺ are taken directly from the area percentages of HPLC measurement with a UV detector:

<1% compound with n=2, <1% compound with n=3, 97% compound with n=4, 1% compound with n=5, <1% compound with n=6.

The product has a slight intrinsic odor.

The mixture of 10 g of polysulfide mixture with 100 ml of water, after being refluxed for 30 min and cooled down to 25° C., exhibited a pH of 3.5 and a conductivity of 0.1 mS/cm.

### EXAMPLE 4

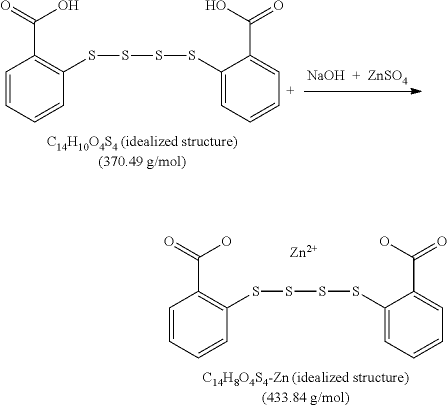 [SMILES: O=C(O)c1ccccc1SSSSc1ccccc1C(=O)O ; O=C(O)c1ccccc1SSSSc1ccccc1C(=O)O ; [Zn+2]]

Apparatus: 2000 ml four-neck flask with thermometer, dropping funnel with pressure equalizer, reflux condenser with gas outlet attachment (bubble counter) and hose, stirrer, pH electrode

Initial charge: 74.1 g of example 1 polysulfide mixture, 700 ml of water

Feed: 35.9 g=0.2 mol of ZnSO₄xH₂O (from Aldrich, 100%) dissolved in 300 ml of water

Feed: 160.0 g=0.4 mol of NaOH solution (10%)

The nitrogen-purged apparatus is initially charged with the water and the polysulfide mixture from example 1 and heated to 95-100° C. The ZnSO₄ solution is then added dropwise to the mixture present under a nitrogen flow at a temperature of 95-100° C. within about 1 h. The mixture is stirred for a further 1 h. This is followed by the metered addition of the NaOH solution at 95-100° C. within about 1 h. The mixture is stirred at about 100° C. for a further 1 h. After cooling, the product is filtered with suction through a D4 frit and washed with 500 ml portions of water until the conductivity of the washing water is <0.3 mS/cm. The product is dried at 50° C. in a vacuum drying cabinet.

Yield: 79.3 g (91.4%) of a polysulfide mixture of the idealized formula

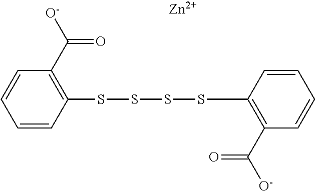 [SMILES: [Zn+2] ; O=C([O-])c1ccccc1SSSSc1ccccc1C(=O)[O-]]

Elemental analysis: C: 37.8% H: 2.1% O: 16.0% S: 29.7% Zn: 16% Cl: 110 ppm

The product has a slight intrinsic odor.

The mixture of 10 g of polysulfide mixture with 100 ml of water, after being refluxed for 30 min and cooled down to 25° C., exhibited a pH of 5.9 and a conductivity of 0.6 mS/cm.

### EXAMPLE 5

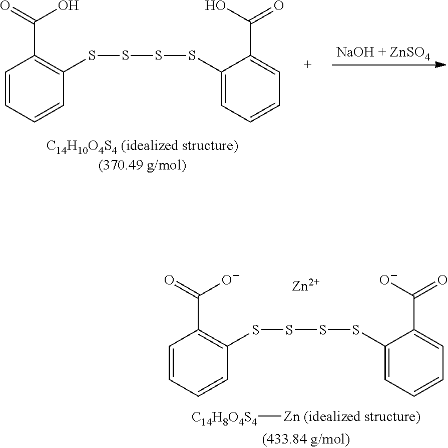

Apparatus: 2000 ml four-neck flask with thermometer, dropping funnel with pressure equalizer, reflux condenser with gas outlet attachment (bubble counter) and hose, stirrer, pH electrode

Initial charge: 74.1 g of example 1 polysulfide mixture, 700 ml of water

Feed: 35.9 g=0.2 mol of ZnSO₄xH₂O (from Aldrich, 100%) dissolved in 300 ml of water

Feed: 160.0 g=0.4 mol of NaOH solution (10%)

The nitrogen-purged apparatus is initially charged with the water and the polysulfide mixture from example 1 at 20-25° C. The ZnSO₄ solution is then added dropwise to the mixture present under a nitrogen flow at a temperature of 20-25° C. within about 1 h. The mixture is stirred for a further 1 h. This is followed by the metered addition of the NaOH solution at 20-25° C. within about 1 h. Then the mixture is heated to gentle reflux and stirred at about 100° C. for a further 1 h. After cooling, the product is filtered with suction through a D4 frit and washed with 500 ml portions of water until the conductivity of the washing water is <0.3 mS/cm. The product is dried at 50° C. in a vacuum drying cabinet.

Yield: 79.3 g (91.0%) of a polysulfide mixture of the idealized formula

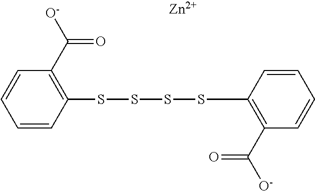 [SMILES: [Zn+2] ; O=C([O-])c1ccccc1SSSSc1ccccc1C(=O)[O-]]

Elemental analysis: C: 38.5% H: 2.2% O: 16.0% S: 29.7% Zn: 14% Cl: 80 ppm

The product has a slight intrinsic odor.

The mixture of 10 g of polysulfide mixture with 100 ml of water, after being refluxed for 30 min and cooled down to 25° C., exhibited a pH of 6.0 and a conductivity of 0.4 mS/cm.

### Production of Rubber Mixtures and Rubber Vulcanizates

The rubber formulations listed in table 1 were each mixed by the multistage process described below.

### 1st Mixing Stage:

BUNA® CB 24 and BUNA® VSL 5025-2 were initially charged in an internal mixer and mixed for about 30 seconds
Addition of two thirds of VULKASIL® S, two thirds of SI® 69, two thirds of the total amount of inventive polysulfides of the formula (I), mix for about 60 seconds
Addition of one third of VULKASIL® S, one third of SI® 69, one third of the total amount of inventive polysulfides of the formula (I) and TUDALEN 1849-1, mix for about 60 seconds

Addition of CORAX® N 339, EDENOR® C 18 98-100, VULKANOX® 4020/LG, VULKANOX® HS/LG, ZINKWEISS ROTSIEGEL and ANTILUX® 654, mix for about 60 seconds. The mixing was effected at a temperature of 150° C.

### 2nd Mixing Stage:

On completion of the first mixing stage, the mix was taken up by a downstream roller system and formed to a sheet and stored at room temperature for 24 hours. The processing temperatures here were below 60° C.

### 3rd Mixing Stage:

The third mixing stage is a further mastication at 150° C. in a kneader.

### 4th Mixing Stage:

Addition of the additives MAHLSCHWEFEL 90/95 CHANCEL, VULKACIT® CZ/C, VULKACIT® D/C on a roller at temperatures below 80° C.

The rubber mixtures were subsequently fully vulcanized at 170° C. The properties of the rubber preparations produced and the vulcanizates thereof are reported in table 2.

TABLE 1
Rubber formulation

| | | Rubber | Rubber | Rubber | Rubber | Rubber |
|---|---|---|---|---|---|---|
| | | formulation | formulation | formulation | formulation | formulation |
| | Reference | 1 | 2 | 3 | 4 | 5 |
| BUNA CB 24 | 30 | 30 | 30 | 30 | 30 | 30 |
| BUNA VSL 5025-2 | 96 | 96 | 96 | 96 | 96 | 96 |
| CORAX N 339 | 6.4 | 6.4 | 6.4 | 6.4 | 6.4 | 6.4 |
| VULKASIL S | 80 | 80 | 80 | 80 | 80 | 80 |
| TUDALEN 1849-1 | 8 | 8 | 8 | 8 | 8 | 8 |
| EDENOR C 18 98-100 | 1 | 1 | 1 | 1 | 1 | 1 |
| VULKANOX 4020/LG | 1 | 1 | 1 | 1 | 1 | 1 |
| VULKANOX HS/LG | 1 | 1 | 1 | 1 | 1 | 1 |
| ZINKWEISS | 2.5 | 2.5 | 2.5 | 2.5 | 2.5 | 2.5 |
| ROTSIEGEL | | | | | | |
| ANTILUX 654 | 1.5 | 1.5 | 1.5 | 1.5 | 1.5 | 1.5 |
| SI 69 | 6.4 | 6.4 | 6.4 | 6.4 | 6.4 | 6.4 |
| VULKACIT D/C | 2 | 2 | 2 | 2 | 2 | 2 |
| VULKACIT CZ/C | 1.5 | 1.5 | 1.5 | 1.5 | 1.5 | 1.5 |
| MAHLSCHWEFEL | 1.5 | 1.5 | 1.5 | 1.5 | 1.5 | 1.5 |
| 90/95 CHANCEL | | | | | | |
| Example 1 compound | | 1 | | | | |
| Example 2 compound | | | 1 | | | |
| Example 3 compound | | | | 1 | | |
| Example 4 compound | | | | | 1 | |
| Example 5 compound | | | | | | 1 |

Amounts stated in phr (parts by weight per 100 parts of rubber)

| Trade name | Details | Manufacturer/distributor |
|---|---|---|
| BUNA CB 24 | BR | Lanxess Deutschland |
| | | GmbH |
| BUNA VSL 5025-2 | SBR | Lanxess Deutschland |
| | | GmbH |
| CORAX N 339 | carbon black | Degussa-Evonik GmbH |
| VULKASIL S | silica | Lanxess Deutschland |
| | | GmbH |
| TUDALEN 1849-1 | mineral oil | Hansen&Rosenthal KG |
| EDENOR C | stearic acid | Cognis Deutschland |
| 18 98-100 | | GmbH |
| VULKANOX | N-1,3-dimethylbutyl- | Lanxess Deutschland |
| 4020/LG | N-phenyl-p- | GmbH |
| | phenylenediamine | |
| VULKANOX | 2,2,4-trimethyl- | Lanxess Deutschland |
| HS/LG | 1,2-dihydroquinoline | GmbH |
| | polymerized | |
| ZINKWEISS | zinc oxide | Grillo Zinkoxid GmbH |
| ROTSIEGEL | | |
| ANTILUX 654 | light stabilizer wax | RheinChemie Rheinau |
| | | GmbH |
| SI 69 | bis(triethoxysilylpropyl) | Evonik Industries |
| | tetrasulfide | |
| VULKACIT D/C | 1,3-diphenylguanidine | Lanxess Deutschland |
| | | GmbH |
| VULKACIT CZ/C | N-cyclonexyl-2- | Lanxess Deutschland |
| | benzothiazole- | GmbH |
| | sulfenamide | |
| MAHLSCHWEFEL | sulfur | Solvay Deutschland |
| 90/95 CHANCEL | | GmbH |

TABLE 2
Summary of results

| | | | | Rubber | Rubber | Rubber | Rubber |
|---|---|---|---|---|---|---|---|
| | | | | formulation | formulation | formulation | formulation |
| Parameter | Unit | DIN | Reference | 1 | 2 | 3 | 5 |
| Mooney | [ME] | 53523 | 95 | 92 | 84 | 91 | 89 |
| viscosity (ML | | | | | | | |
| 1 + 4) | | | | | | | |
| Mooney scorch | sec | ASTM D | 1253 | 1228 | 1020 | 972 | 960 |
| at 130° C. (t5) | | 5289-95 | | | | | |
| Vulcanization at | sec | 53529 | 1417 | 1315 | 1475 | 1844 | 1580 |
| 170° C. (t95) | | | | | | | |
| Shore A | [Shore A] | 53505 | 66 | 73 | 67 | 69 | 68 |
| hardness at | | | | | | | |
| 23° C. | | | | | | | |
| 300 modulus | MPa | 53504 | 15 | 18 | 16 | 17 | 17 |
| Elongation at | % | 53504 | 349 | 308 | 361 | 302 | 314 |
| break | | | | | | | |
| Tensile strength | MPa | 53504 | 19 | 18 | 20 | 18 | 18 |
| Abrasion | mm³ | 53516 | 74 | 93 | 83 | 77 | 72 |
| Rolling | — | | 0.133 | 0.154 | 0.093 | 0.094 | 0.090 |
| resistance (tan | | | | | | | |
| δ (60° C.)) | | | | | | | |

The tested vulcanizates comprising the inventive rubber mixtures 2, 3 and 5, compared to the reference, show elevated hardness values and 300 modulus values, improved rolling resistance and lower Mooney viscosity. Compared to rubber mixture 1 comprising a compound of the formula (I) having a relatively high chlorine content and relatively high residual acidity, the inventive rubber mixtures 2, 3 and 5 show a distinct improvement in terms of abrasion and rolling resistance, with a maintained or reduced Mooney viscosity.

What is claimed is:

1. Polysulfides of the formula (I) 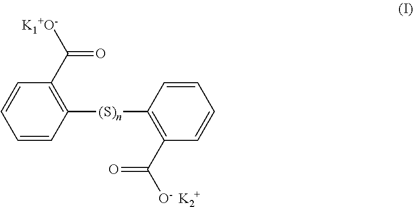
where:
the cations K₁⁺ and K₂⁺ are each independently any monovalent cation or a fraction of any polyvalent cation which corresponds to a positive charge of one, and
a portion of the polysulfides of the formula (I) have n=4, and an additional portion of the polysulfides of the formula (I) have n=2, 3, 5 and/or 6, and the portion of polysulfides of the formula (I) with n=4, based on the total amount of polysulfides of the formula (I), is more than 80%.

2. The polysulfides as claimed in claim 1, wherein the portion of polysulfides of the formula (I) with n=4, based on the total amount of polysulfides of the formula (I), is more than 90%.

3. The polysulfides as claimed in claim 1, wherein the cations K₁⁺ and K₂⁺ are each independently H⁺, an alkali metal cation, ½ alkaline earth metal cation, a fraction of a rare earth metal cation which corresponds to a positive charge of one, or ½ Zn²⁺.

4. The polysulfides as claimed in claim 1, wherein a mixture of 10 g of polysulfides of the formula (I) in 100 ml of water, after reflux for 30 min and subsequent cooling to 25° C. within 60 min, has a conductivity of <5 mS/cm.

5. The polysulfides as claimed in claim 1, wherein a mixture of 10 g of polysulfides of the formula (I) and 100 ml of water, after being refluxed at standard pressure for 30 min and then cooled down to 25° C. within 60 min, has a pH of >2.

6. The polysulfides as claimed in claim 1, wherein the polysulfides of the formula (I) have a total chlorine content <1%.

7. Polysulfides as claimed in claim 1, wherein the cations K₁⁺ and K₂⁺ are each independently H⁺, or ½ Zn²⁺, and the polysulfides have a total chlorine content <0.3%.

8. Polysulfides as claimed in claim 1, wherein the cations K₁⁺ and K₂⁺ are each independently H⁺, or ½ Zn²⁺, the polysulfides have a total chlorine content <0.03%, and a mixture of 10 g of polysulfides in 100 ml of water, after reflux for 30 min and subsequent cooling to 25° C. within 60 min, has a pH >2 and a conductivity of <1 mS/cm.

9. The polysulfides as claimed in claim 1, wherein:
K₁⁺ and K₂⁺ are each H⁺,
the melting range of the polysulfides ends at at least 300° C., where the end of the melting range is determined at a heating rate of 1° C./min, starting from 290° C.; and
the polysulfides have a dominant signal in the x-ray diffractogram (Cu K-alpha radiation) at a diffraction angle 2theta° of 27.2+/−0.1.

10. The polysdulfides as claimed in claim 1, wherein:
K₁⁺ and K₂⁺ are each H⁺,
the polysulfides have a total chlorine content of <0.03%;
96-99% of the polysulfides of the formula (I) are polysulfides of the formula (I) with n=4;
the polysulfides have a melting range ending at at least 304° C., where the end of the melting range is determined at a heating rate of 1° C./min, starting from 290° C.; and
the polysulfides have a dominant signal in the x-ray diffractogram (Cu K-alpha radiation) at a diffraction angle 2theta° of 27.2 +/−0.1; and
a mixture of 10 g of the polysulfides of the formula (I) in 100 ml of water, after reflux for 30 min and subsequent cooling to 25° C. within 60 min, has a pH of 3.4-6.2 and a conductivity of <1 mS/cm.

11. A process for preparing the polysulfides as claimed in claim 1, the process comprising:
a) reacting 2-mercaptobenzoic acid with S₂Cl₂ to give the polysulfides of the formula (I) where K₁⁺ and K₂⁺ are each H⁺ and n is 2, 3, 4, 5 and/or 6, and
at least one of:
b1) contacting the resulting polysulfides of the formula (I) with water or a mixture of water and an inert organic medium, and heating to a temperature of >60° C., and
b2) contacting the resulting polysulfides of the formula (I) with a solution of metal salts of the cations K₁⁺ and K₂⁺ at a temperatures >60° C., and treating the polysulfides at temperatures of 80° C.-120° C.

12. A rubber mixture comprising:
at least one rubber,
at least one filler, and
the polysulfides as claimed in claim 1.

13. The rubber mixture as claimed in claim 12, wherein:
the at least one filler comprises one or more silica-based fillers, and a proportion by weight of silica-based fillers is at least 10% of a total filler content, and
the rubber mixture additionally contains a sulfur-containing alkoxysilane.

14. The rubber mixture as claimed in claim 12, wherein the rubber mixture additionally comprises one or more crosslinkers.

15. The rubber mixture as claimed in claim 12, wherein the rubber mixture comprises 0.1 to 15 phr of the polysulfides.

16. The rubber mixture as claimed in claim 12, wherein the at least one rubber comprises at least one SBR rubber and at least one BR rubber in a weight ratio of SBA rubber:BR rubber of 60:40 to 90:10.

17. The rubber mixture as claimed in claim 16, wherein:
the at least one rubber further comprises at least one NR rubber, and a weight ratio of SBR rubber to BR rubber to NR rubber is 60 to 85:10 to 35:5 to 20;
the filler comprises one or more silica-based fillers, and the proportion by weight of silica-based fillers is at least 80% of a total filler content;
the rubber mixture additionally contains a sulfur-containing alkoxysilane, and
the rubber mixture comprises 0.7 to 15 phr of the polysulfides.

18. A process for producing the rubber mixtures as claimed in claim 12, the process comprising mixing the at least one rubber, the at least one filler, and the polysulfides by a multistage mixing process in which the polysulfides are added in a first stage of the mixing process, and one or more crosslinkers are added in a later mixing stage subsequent to the first stage.

19. A process for producing vulcanizates, the process comprising vulcanizing the rubber mixture as claimed in claim 10 at blend temperatures of 100 to 200° C.

20. A vulcanizate obtained by the process of claim 19.
